(19) United States
(12) Patent Application Publication
(10) Pub. No.: US 2006/0020045 A1
(43) Pub. Date: Jan. 26, 2006

(54) Methods and compositions for treating inflammatory disorders of the gastrointestinal tract

(76) Inventor: Roger Berlin, Mendham, NJ (US)

Correspondence Address: WYETH;PATENT LAW GROUP, 5 GIRALDA FARMS, MADISON, NJ, 07940, US

(21) Appl. No.: 10/898,896

(22) Filed: Jul. 26, 2004

(51) Int. Cl. A61K 31/045

(52) U.S. Cl. 514/724

(57) ABSTRACT

The present invention provides compositions and methods for treating or preventing the development of inflammatory disorder of the gastrointestinal tract in a subject, comprising administering to a subject therapeutically effective amount of a biologically active mixture of high purity, high molecular weight straight chain primary aliphatic alcohols (referred to collectively herein as policosanol). Inflammatory conditions that can be addressed by the compositions and methods of this invention include colorectal cancer, neoplasm of the colon, neoplasm of the rectum, ulcerative colitis, adenomatous polyps, familial polyposis, stomach cancer, and combinations thereof.

### BACKGROUND OF THE INVENTION

1. Field of the Invention

The present invention relates to therapeutic compositions and methods for treating, ameliorating, or inhibiting the development of an inflammatory condition of the gastrointestinal tract in a subject, including humans. More particularly the invention pertains to methods of treating or preventing the development of inflammatory condition of the gastrointestinal tract such as colorectal cancer, neoplasm of the colon, neoplasm of the rectum, ulcerative colitis, adenomatous polyps, familial polyposis, stomach cancer, and combinations thereof, by administering to a subject therapeutically effective amount of a biologically active mixture of high purity, high molecular weight straight chain primary aliphatic alcohols (referred to collectively herein as policosanol).

2. Description of the State of Art

Colonic polyps occur in both a familial pattern (familial adenomatous polyps; FAP) and sporadically. FAP afflicts approximately 25,000 patients in the US; while it is estimated that sporadic adenomatous polyps (SAP) occur in approximately 2 million people per year in the US alone. All these patients are at risk for developing adenocarcinoma of the colon. In the case of FAP, that risk is virtually 100% and these patients usually undergo a colectomy at an early age. Patients with sporadic polyps are treated with polypectomy and require periodic colonoscopic examination because of their inherent risk of developing recurrent polyps. In fact, parents and siblings of these patients are also at increased risk for developing colorectal cancer.

Colon cancer currently accounts for 11% of all deaths due to malignancy annually in the United States. With an incidence of 62 per 100,000 and a prevalence of 300 per 100,000, the disease is currently the third leading cause of death in men and the fourth leading cause of death in women. Colon cancer has a particularly poor five-year survival rate of less than 50%, due to the late stage at which diagnosis is generally made. The currently favored treatment, surgery combined with chemotherapy, has failed to increase this survival rate. What is needed is a safe and effective preventive therapy which could be initiated early in patient populations known to be at an increased risk of developing colon cancer.

It is also well documented that chronic gastrointestinal inflammatory diseases such as ulcerative colitis (Lennard-Jones et al. (1977) Gastroenterology 73:1280-1285), Crohn's disease (Farmer et al. (1971) Cancer 28:289-295), and chronic atrophic gastritis (Sipponen et al. (1983) Cancer 52:1062-1067) are associated with an increased risk of subsequent gastrointestinal cancer. Although the mechanisms are not yet proven, three important intersecting pathways which could lead to subsequent transformation, heightened proliferation and malignant invasion occur during multiple acute inflammatory episodes.

Recently, it has been hypothesized that inflammation can increase the risk of cancer (Coussens, et al., Nature, 2002; 420:860-867). Although there is growing evidence from laboratory studies supporting the role of inflammation in the pathogenesis of colorectal cancer, data from epidemiologic studies are sparse. However, inflammation could be particularly important in the pathogenesis of colorectal cancers. Chronic inflammatory bowel diseases, such as ulcerative colitis, have been associated with the increased risk of cancer (Levin, B., Cancer, 1992; 70(5 suppl): 1313-1316). Moreover, several studies have shown a reduced risk of colon cancer with the use of aspirin or other anti-inflammatory agents (Baron, et al., N. Engl. J. Med., 2003; 348:891-899; Sandler, R, et al., N. Engl. J. Med., 2003; 348:883-890).

There is evidence that several non-steroid anti-inflammatory drugs (NSAIDs) are effective in reducing the number of tumor-bearing animals and tumor incidence per animal in rat models of colon carcinogenesis (Narisawa et al. (1981) Cancer Res. 41:1954-1957; (Pollard et al. (1983) Cancer Lett. 21:57-61; Moorghen et al. (1988) J. Pathol. 156:341-347; Reddy et al. (1993) Gastroenterology 104:A443) where up to a 70% reduction in tumor incidence has been noted at doses of 80% of the maximum tolerated dose. In a study of dimethylhydrazine-induced colon carcinogenesis, sulindac was observed to reduce tumor incidence only when present during carcinogen administration, but not if given 17 weeks following the carcinogen (Moorghen et al. (1988) J. Pathol. 156:341-347).

In several retrospective studies, aspirin intake has been assessed as a chemopreventive therapy in ensuing colon cancer incidence. The results of these studies have ranged from a halving (Kune et al. (1988) Cancer Res. 48:4399-4404) of the risk of colon cancer development to a 50% increased risk (Paganini-Hill et al. (1991) J. Natl. Cancer Inst. 83:1182-1183). Unfortunately, NSAIDs also have a disturbing and serious gastrointestinal side effect profile which may preclude their chronic use.

A number of proximal mediators of the inflammatory response have been identified. In particular, C-reactive protein (CRP) has recently gained recognition as a marker for inflammatory conditions (see U.S. Pat. No. 6,040,147). C-reactive protein (CRP) is an acute-phase protein produced primarily in the liver in response to stimulation by interleukin 6 (IL-6). CRP has been identified as a marker of inflammation circulating in the blood and has been associated with increased risk of heart disease. Recently, it has been suggested that CRP could also be used to identify a person's risk of developing colon cancer, due to the increasing evidence identifying inflammation as a risk factor for the disease. According to the results of a study at Johns Hopkins University (Erlinger, et al., JAMA, 2004; 291:585-590) conducted over an 11-year period, people with higher levels of CRP in the blood (a median of 2.44 milligrams per liter) were more likely to develop colorectal cancers than those with low levels of CRP (a median of 1.94 mg/L). The study also found that 1) the odds of developing colorectal cancers increased progressively with higher concentrations of CRP; 2) among nonsmokers, those in the highest quartile were 2.5 times as likely to develop colorectal cancer, as those in the lowest quartile; and 3) those who had taken either aspirin or nonsteroidal anti-inflammatory agents within the 48 hours prior to blood draw had a reduced risk of colorectal cancer.

A mixture of high purity, high molecular weight straight chain aliphatic alcohols (collectively referred to herein as policosanol) has garnered much interest in recent years as a natural supplement for its cholesterol-lowering effects (Gouni-Berthold, et al., Am. Heart J., 143(2):356-365 (2002)). The main constituents of policosanol are tetracosanol, hexacosanol, octacosanol, and triacontanol, while eicosanol, docosanol, heptacosanol, nonacosanol, dotriacontanol, tetratriacontanol, and hexatriacontanol make up the remaining minor constituents of the straight chain aliphatic alcohols. There is a significant body of evidence demonstrating the benefits of policosanol with respect to cardiovascular disease. In the mid to late nineties, one research group proposed that policosanol was able to reduce endothelial damage by inhibiting the production of foam cells (Noa, et al., J. Pharm. Pharmacol., 48(3):306-309 (1996); Noa, et al., J. Pharm. Pharmacol., 49(10):999-1002 (1997)). Foam cells are macrophages that can migrate into the endothelium of the blood vessels and contribute to atherosclerotic plaque formation (Physicians' Desk Reference, 50 ed., Montvale, N.J.: Medical Economics Company; 2002.). Other researchers believe policosanol has a modulating effect on HMG-CoA reductase, the rate-controlling enzyme in cholesterol biosynthesis, but the precise mechanism remains unclear (Menendez, et al., Biol. Res., 27(3-4):199-203 (1994); Menendez, et al., Biol. Res., 29(2):253-257 (1996); and Menendez, et al., Arch. Med. Res., 32(1):8-12 (2001)). Still other investigators believe policosanol may inhibit cholesterol synthesis in the liver at a step before mevalonate production, but total inhibition of the HMG-CoA reductase is doubtful (Gouni-Berthold, et al., Am. Heart J., 143(2):356-365 (2002)). More recent work suggests policosanol inhibits LDL cholesterol oxidation (Menendez, et al., Can. J. Physiol. Pharmacol., 80(1): 13-21 (2002); Menendez, et al., Br. J. Clin. Pharmacol., 50(3):255-262 (2000)). This was revealed when markers of peroxidation, such as thiobarbituric acid reactive substances (TBARS), and malondialdehyde (MDA) were lower in the cultures treated with policosanol. Oxidation of LDL cholesterol has been linked to heart disease and was the recent cover story in Scientific American magazine (Physicians' Desk Reference, 50 ed., Montvale, N.J.: Medical Economics Company; 2002).

### SUMMARY OF THE INVENTION

The present invention provides compositions and methods for treating, ameliorating, or inhibiting the development of an inflammatory condition of the gastrointestinal tract in a subject, comprising administering a therapeutically effective amount of a formulation of the present invention. The formulation of the present invention comprises a mixture of high purity, high molecular weight straight chain primary aliphatic alcohols, wherein the composition comprises from about 1% to about 90% by weight policosanol. The formulation may further comprise from 0% to about 65% by weight of pharmaceutically acceptable formulation aids including, but not limited to diluents, stabilizers, binders, buffers, lubricants, coating agents, preservatives, emulsifiers and/or suspension agents. In one embodiment of this invention, the policosanol comprises at least one high molecular weight straight chain primary aliphatic alcohol selected from 20 to 36 carbon atoms.

The daily dosage is preferably between 1 and 100 mg of policosanol (more preferably between about 3 and 20 mg) and is intended for administration in any type or form.

In another embodiment, the policosanol composition of this invention comprises 1-tetracosanol, 1-hexacosanol, 1-heptacosanol, 1-octacosanol, 1-triacontanol, 1-dotriacontanol and 1-tetratriacontanol.

In yet another embodiment, the composition of the present invention comprises policosanol having the following quantitative composition:

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 0-5% |
| 1-octacosanol (C₂₈) | 5-80% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |
| 1-hexatriacontanol (C₃₆) | 0-5% |

The present invention further provides kits having one or more containers comprising a therapeutic composition of the present invention and a suitable excipient as described herein and a set of instructions, generally written instructions although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

Inflammatory conditions of the gastrointestinal tract that can be addressed by compositions and methods of the present invention include, but are not limited to, colorectal cancer, neoplasm of the colon, neoplasm of the rectum, ulcerative colitis, adenomatous polyps, familial polyposis, stomach cancer, and combinations thereof.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### DETAILED DESCRIPTION OF THE INVENTION

The present invention provides compositions and methods for treating, ameliorating, or inhibiting the development of an inflammatory condition of the gastrointestinal tract in a subject, comprising administering a therapeutically effective amount of a composition comprising a mixture of high purity, high molecular weight straight chain primary aliphatic alcohols (referred collectively herein to as policosanol) to a subject in need thereof.

The policosanol used in the present invention can be derived from any suitable source, each source being associated with a policosanol of particular characteristics, usually in terms of the relative proportions of its primary aliphatic alcohol components. For example, policosanol can be extracted and purified from a wide array of starting materials, such as, but not limited to, pela bug, natural waxes such as but not limited to, beeswax, camauba wax, and candellia wax; bee pollen; oils such as but not limited to, peanut oil, sesame oil, cod liver oil, rice bran oil, oat oil, and rosemary needles oil; and powders such as but not limited to rice bran, containing primarily natural esters of aliphatic alcohols with caboxylic acids. Consequently, the quantitative compositions of policosanol can vary depending on the extraction process and starting materials that are used in its production. In one embodiment, the policosanol has the quantitative composition set forth in Table 1.

TABLE I

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 0-5% |
| 1-octacosanol (C₂₈) | 5-80% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |
| 1-hexatriacontanol (C₃₆) | 0-5% |

U.S. Pat. Nos. 5,663,156; 5,856,316; 6,197,832; 6,225,354; and 6,596,776, all of which are incorporated herein by reference, disclose policosanol compositions that are specific to the starting material and extraction processes used. It should be noted that while any commercially available policosanol or any of the policosanols disclosed in the above-referenced patents are suitable for use in the present invention, for purposes of the remainder of this discussion the policosanol and methodologies disclosed in U.S. Pat. No. 6,596,776 will be referenced. Specifically, in one embodiment the policosanol used in the present invention is obtained from beeswax and has the formulation set forth in Table II.

TABLE II

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 13-28% |
| 1-hexacosanol (C₂₆) | 5-30% |
| 1-heptacosanol (C₂₇) | 0-5% |
| 1-octacosanol (C₂₈) | 15-25% |
| 1-triacontanol (C₃₀) | 25-40% |
| 1-dotriacontanol (C₃₂) | 5-15% |
| 1-tetratriacontanol (C₃₄) | 0-5% |

The process used to isolate the policosanol composition described in Table II is incorporated herein by reference and is briefly described as follows. Beeswax is initially subjected to a homogenous phase saponification step, after which the saponified beeswax is dried and ground to a particle mesh size of 100-500 microns. Alternatively, unsaponified beeswax of varying purity may be used as the starting material and is initially dried and ground to a particle mesh size of 100-200 microns. The particles of saponified or unsaponified beeswax are placed into a conventional solid-liquid extractor and a hot organic solvent is introduced and contacted with the beeswax particles. The suspension is mixed and then hot-filtered to remove any solids.

The resulting extract is then maintained within the temperature range of 2° C. to 100° C., causing the aliphatic alcohols to solidify and form a suspension. The suspension is filtered and the first solids are recovered and air dried. The dried solids are then sent to a purifier where they are contacted with and dissolved in a second hot solvent and hot-filtered. This solution is then cooled and the second solids are collected and dried under vacuum. The dried solids obtained from the second purification step are contacted with a third hot organic solvent, which dissolves the solids. This solution is hot-filtered and chilled, and the third solids are collected, dried, and powdered to provide the final product disclosed in Table II.

After the particles are dried, they can be formulated into a conventional pharmaceutical formulation for administration. The formulations of the present invention comprise a therapeutically effective amount policosanol in the range between about 0.5 and 99%.

Policosanol is extremely well tolerated. In animal toxicity studies, doses up to 500 mg/kg/day, a dose that is 1500 times the normal human dose of 20 mg/day, have shown no negative effects on carcinogenesis, reproduction, growth, or development. In one embodiment, a total dose of policosanol administered to a subject according to the present invention is in the range from about 1 mg to 100 mg per day. In another embodiment, a total dose of policosanol between about 5 mg to 40 mg per day is administered. In yet another embodiment, a total dose of policosanol between about 10 to 20 mg per day is administered.

As used herein, a “therapeutically effective amount” is an amount that will provide the desired activity without significant side-effect. For example, by way of illustration, the amount of policosanol that will be effective in the treatment of colorectal cancer will depend in part on the severity of the cancer, and can be determined by standard clinical techniques. In addition, in vitro or in vivo assays may optionally be employed to help identify optimal dosage ranges. Effective doses may be extrapolated from dose-response curves derived from in vitro or animal model test systems. The precise dosage level should be determined by the attending physician or other health care provider and will depend upon well-known factors, including route of administration, and the age, body weight, sex and general health of the individual; the nature, severity and clinical stage of the tumor(s); and the use (or not) of concomitant therapies. Of course, the skilled person will realize that divided and partial doses are also within the scope of the invention. The responses to treatment in solid tumors can be evaluated using guidelines such as those published by the World Health Organization in 1979 (WHO handbook for reporting results of cancer treatment (1979), World Health Organization Offset Publication No. 48); by Miller et al. in 1981 (Miller et al. (1981) Cancer 47:207-214); and the response evaluation criteria in solid tumors (RECIST) by Therasse et al. in 2000 (Therasse et al. (2000) J. Natl. Cancer Inst. 92:205-216). Other evidence of a therapeutically effective amount is provided by, for example, when the administration of a policosanol composition of this invention reduces the overall tumor burden, results in an objective response, slows tumor progression, prevents tumor recurrence, prevents the appearance of new tumor lesions, results in a partial or complete response in a tumor lesion, or results in a therapeutic benefit to the subject.

Inflammatory disorders of the gastrointestinal tract include, but are not limited to, colorectal cancer, neoplasm of the colon, neoplasm of the rectum, ulcerative colitis, adenomatous polyps, familial polyposis, stomach cancer, and combinations thereof.

The term “colorectal cancer” is meant to include the well accepted medical definition that defines colorectal cancer as a medical condition characterized by cancer of cells of the intestinal tract below the small intestine (i.e. the large intestine (colon), including the cecum, ascending colon, transverse colon, descending colon, and sigmoid colon, and rectum). Additionally, as used herein, the term “colorectal cancer” is meant to further include medical conditions which are characterized by cancer of cells of the duodenum and small intestine (jejunum and ileum). The term “colorectal cancer” also includes adenocarcinoma and carcinoma of the colon whether in a primary site or metastasized.

A neoplasm, or tumor, is an abnormal, unregulated, and disorganized proliferation of cell growth, and is generally referred to as cancer. A neoplasm is malignant, or cancerous, if it has properties of destructive growth, invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphotics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

The term “treatment” or “treating” includes, but is not limited to, any treatment of a disease or disorder, in a mammal, including, but not limited to: preventing or protecting against the disease or disorder that causes the clinical symptoms of the disease; inhibiting the disease, that is, arresting or suppressing the development of clinical symptoms; and/or relieving the disease, that is, causing the regression of clinical symptoms. For example, in one embodiment of this invention the treatment of colorectal cancer includes the prevention of the onset of cancer; the inhibition of the growth of existing cancer; the prevention of the reoccurrence of cancer; and the total arrest of cancer. The prevention of the reoccurrence of cancer includes, but is not limited to, cancer sites and surrounding tissue which have previously been treated by radiation, chemotherapy, surgery or other techniques. Prevention also includes circumstances of prophylaxes where the onset of oncogenesis or tumorigenesis is not evidenced in the patient such as circumstances where a predisposition for cancer is identified whether determined by genetic screening, for example, or otherwise.

The term “amelioration” includes, but is not limited to, the prevention, reduction or palliation of a state, or improvement of the state of a subject. Amelioration includes, but does not require complete recovery or complete prevention of a symptom or condition. More specifically, amelioration may be considered to be at least about 30%, at least about 50%, at least about 70%, at least about 80%, and at least about 90% reduction in the symptoms associated with an inflammatory disorder of the gastrointestinal tract.

A “subject” includes both humans and other animals (particularly mammals) that receive either prophylactic or therapeutic treatment according to this invention.

The pharmaceutical formulations of the present invention can contain as an active ingredients from about 0.5 to about 95.0% wt of policosanol. This dosage is obtained by mixing the policosanol with one or more excipients, including, but not limited to agglutinants, disintegrators, lubricants, sliders or fillers. For example, specific excipients include, but are not limited to, lactose, corn starch, saccharose, magnesium stearate, microcrystalline cellulose, sodium croscarmellose gelatin, cellulose acetophtalate, titanium dioxide, special talc for tablets and polyethylene glycol. Other suitable excipients are well known in the art. The formulation may further comprise from 0% to about 65% by weight of pharmaceutically acceptable formulation aids, including but not limited to diluents, stabilizers, binders, buffers, lubricants, coating agents, preservatives, emulsifiers and/or suspension agents.

A composition of the present invention may be combined or used in combination with other methods known in the art for treating, ameliorating, or preventing the development of an inflammatory disorder of the gastrointestinal tract such as cancers, neoplasms, or polyps. For example, a method according to this invention can include administering policosanol and at least one additional chemotherapy agent, wherein said policosanol and said additional chemotherapy agent are administered as an admixture, separately and simultaneously, or separately in any order. Alternatively, policosanol can be administered before, during, or after conventional radiation therapy.

The amount of policosanol in a formulation of this invention that is combined with one or more excipients to produce a single dosage form will necessarily vary depending upon the host treated and the particular route of administration. For example, a formulation intended for oral administration to humans will may contain, for example, from 1-30 g of policosanol compounded with an appropriate and convenient amount of excipients which may vary from about 5 to about 95 percent by weight of the total composition. Dosage unit forms will generally contain about 1-30 g of policosanol. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In order to use the compositions of this invention for the therapeutic treatment (including prophylactic treatment) of a subject in need thereof according to the methods of this invention, policosanol is formulated in accordance with standard pharmaceutical practice as a pharmaceutical formulation as discussed above. According to this aspect of the invention there is provided a pharmaceutical composition comprising policosanol in association with a pharmaceutically acceptable diluent or carrier, wherein the policosanol is present in an amount for effectively treating or preventing inflammatory disorders of the gastrointestinal tract in an subject.

The formulations of the present invention can be administered to a subject by any available and effective delivery system including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and in a vehicle as desired, such as a depot or a controlled release formulation.

For example, a pharmaceutically acceptable formulation of the composition of the present invention may be formulated for parenteral administration, e.g., for intravenous, subcutaneous, or intramuscular injection. For an injectable formulation, policosanol may be combined with a sterile aqueous solution which is preferably isotonic with the blood of the subject. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by methods known in the art. The formulations may be present in unit or multi-dose containers, such as sealed ampules or vials. The formulation may be delivered by any mode of injection, including, without limitation, epifascial, intracutaneous, intramuscular, intravascular, intravenous, parenchymatous, subcutaneous, oral or nasal preparations (see, for example, U.S. Pat. No. 5,958,877, which is specifically incorporated herein by reference).

Pharmaceutical compositions may also be in the form of a sterile injectable aqueous or oily suspension, which may be formulated according to known procedures using one or more appropriate dispersing or wetting agents and suspending agents. A sterile injectable preparation may also be a sterile injectable solution or suspension in a non-toxic parenterally-acceptable diluent or solvent, for example a solution in 1,3-butanediol.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing), or as vehicles such as a depot or other controlled release formulation.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which policosanol is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain policosanol in finely powdered form together with one or more suspending agents, such as sodium carboxymethylcellulose, methylcellulose, hydroxypropylmethylcel ulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending policosanol in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, wherein policosanol may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing policosanol with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating policosanol with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either policosanol alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense policosanol either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

Controlled Release Formulations

Another aspect of this invention provides methods of treating inflammatory disorders of the gastrointestinal tract by delivering the composition of the present invention to a subject as a controlled release formulation. As used herein, the terms “controlled” “extended” “sustained” or “prolonged” release formulations and methods of the present invention will collectively be referred to as “controlled release” formulations and methods, and include continuous and discontinuous, linear and non-linear release of the composition of the present invention. There are many advantages for a controlled release formulation. Among these are to effectively treat the subject during a period when the subject would not be readily able or willing to periodically ingest the composition of the present invention. The single dose of composition of the present invention preferably is administered via ingestion of one or more controlled release unit dosage forms, so that effective policosanol levels are maintained.

1. Tablets

A useful controlled release tablet is disclosed in U.S. Pat. No. 5,126,145 which is incorporated herein by reference. This tablet comprises, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% policosanol.

More specifically, one such useful controlled release tablet comprises: (a) about 5-20 percent by weight hydroxypropyl methylcellulose having a viscosity of about 10,000 CPS or greater, a substitution rate for the methoxyl group of about 7-30% and a substitution rate for the hydroxypropoxyl group of about 7-20%; (b) about 2-8 percent hydroxypropyl methylcellulose having a viscosity of less than about 100, CPS methyl cellulose, or polyvinyl pyrollidone; (c) about 5-15 percent by weight hydrogenated vegetable oil or stearic acid; and (d) about 30-90% policosanol.

High viscosity water-soluble 2-hydroxypropyl methyl cellulose (HPMC) is particularly preferred for use in the present tablets and in the controlled-release tablet coating, due to its sustaining properties with respect to policosanol release. A particularly preferred high viscosity HMPC has a nominal viscosity, two percent solution, of about 100,000 CPS, methoxyl content of about 19-24, a hydroxypropyl content of about 7-12 percent, and a particle size where at least 90% passes through a USS 100 mesh screen. (Methocel® K100MCR). Low viscosity HPMC is preferred as the binder component of the tablet. A particularly preferred low viscosity HPMC has a methoxyl content of about 20-30%, a hydroxylpropyl content of about 7-12 percent, and a particle size where 100% will pass through a USS No. 30 mesh screen and 99% will pass through a USS 40 mesh screen (Methocel® EIS). In some cases, a portion of the high viscosity HPMC can be replaced by a medium viscosity HPMC, i.e., of about 2000-8,000 cps.

The viscosities reported herein are measured in centipoises (cps or cP), as measured in a 2% by weight aqueous solution of the cellulose either at 20° C. using a rotational viscometer. A “high viscosity” cellulose ether possesses a viscosity of at least about 10,000 cps i.e., about 50,000-100,000 cps. A low-viscosity cellulose ether possesses a viscosity of less than about 100 cps, i.e., about 10-100 cps.

“Water soluble” for purposes of this application means that two grams of powdered cellulose ether can be dispersed by stirring into 100 grams of water at a temperature between 0° C. and 100° C. to provide a substantially clear, stable aqueous composition or dispersion (when the dispersion is brought to 20° C.).

Useful hydrophobic components include natural and synthetic waxes such as beeswax, carnauba wax, paraffin, spermaceti, as well as synthetic waxes, hydrogenated vegetable oils, fatty acids, fatty alcohols and the like.

The controlled release policosanol tablets preferably can be formulated to contain 10 mg, 20 mg or 40 mg of policosanol, and are ingested orally. Preferably, these tablets will release about 10-35 wt % of the total policosanol within about 2 hours in an in vitro dissolution test, and about 40-0 wt-% of the total policosanol in eight hours.

2. Films

This invention further provides a prophylaxis for or method of treating macular disorders in a subject, comprising administering to a subject a biodegradable, biocompatible polymeric film comprising policosanol. The polymeric films are thin compared to their length and breadth. The films typically have a uniform selected thickness between about 60 micrometers and about 5 mm. Films of between about 600 micrometers and 1 mm and between about 1 mm and about 5 mm thick, as well as films between about 60 micrometers and about 1000 micrometers; and between about 60 and about 300 micrometers are useful in the manufacture of therapeutic implants for insertion into a subject's body. The films can be administered to the subject in a manner similar to methods used in adhesion surgeries. For example, a policosanol film formulation can be sprayed or dropped onto a tissue site during surgery, or a formed film can be placed over the selected tissue site. In an alternative embodiment, the film can be used as controlled release coating on a medical device such as a stent.

Either biodegradable or nonbiodegradable polymers may be used to fabricate implants in which the policosanol is uniformly distributed throughout the polymer matrix. A number of suitable biodegradable polymers for use in making the biodegradable films of this invention are known to the art, including polyanhydrides and aliphatic polyesters, preferably polylactic acid (PLA), polyglycolic acid (PGA) and mixtures and copolymers thereof, more preferably 50:50 copolymers of PLA:PGA and most preferably 75:25 copolymers of PLA:PGA. Single enantiomers of PLA may also be used, preferably L-PLA, either alone or in combination with PGA. Polycarbonates, polyfumarates and caprolactones may also be used to make the implants of this invention.

A plasticizer may be incorporated in the biodegradable film to make it softer and more pliable for applications where direct contact with a contoured surface is desired.

The polymeric films of this invention can be formed and used as flat sheets, or can be formed into three-dimensional conformations or “shells” molded to fit the contours of the tissue site into which the film is inserted.

To make the polymeric films of this invention, a suitable polymeric material is selected, depending on the degradation time desired for the film. Selection of such polymeric materials is known to the art. A lower molecular weight, e.g., around 20,000 daltons, 50:50 or 55:45 PLA:PGA copolymer is used when a shorter degradation time is desired. To ensure a selected degradation time, the molecular weights and compositions may be varied as known to the art.

Polymeric films of this invention may be made by dissolving the selected polymeric material in a solvent known to the art, e.g., acetone, chloroform or methylene chloride, using about 20 mL solvent per gram of polymer. The solution is then degassed, preferably under gentle vacuum to remove dissolved air and poured onto a surface, preferably a flat non-stick surface such as BYTAC (Trademark of Norton Performance Plastics, Akron, Ohio) non-stick coated adhesive-backed aluminum foil, glass or TEFLON™ non-stick polymer. The solution is then dried, preferably air-dried, until it is no longer tacky and the liquid appears to be gone. The known density of the polymer may be used to back-calculate the volume of solution needed to produce a film of the desired thickness.

Films may also be made by heat pressing and melt forming/drawing methods known to the art. For example, thicker films can be pressed to form thinner films, and can be drawn out after heating and pulled over forms of the desired shapes, or pulled against a mold by vacuum pressure.

The amount of policosanol to be incorporated into the polymeric films of this invention is an amount effective to show a measurable effect in treating of a macular disorder. The composition of the present invention can be incorporated into the film by various techniques such as by solution methods, suspension methods, or melt pressing.

Solid implants comprising policosanol can also be made into various shapes other than films by injection molding or extrusion techniques. For example, the implant can comprise a core material such as ethylene/vinyl acetate copolymer, and a vinyl acetate content of 20% by weight or more and which functions as a matrix for the policosanol, in a quantity which is sufficient for a controlled release of policosanol, and a membrane which encases the core material and also consists of EVA material and an acetate content of less than 20% by weight. The implant can be obtained, for example, by means of a co-axial extrusion process, a method in which the two EVA polymers are extruded co-axially with the aid of a co-axial extrusion head. The co-axial extrusion process is well known in the art known and need not be described further.

3. Transdermal Patch Device

Transdermal delivery involves delivery of a therapeutic agent through the skin for distribution within the body by circulation of the blood. Transdermal delivery can be compared to continuous, controlled intravenous delivery of a drug using the skin as a port of entry instead of an intravenous needle. The therapeutic agent passes through the outer layers of the skin, diffuses into the capillaries or tiny blood vessels in the skin and then is transported into the main circulatory system.

Transdermal patch devices that can be used to provide a controlled, continuous administration of policosanol through the skin are well known in the art. Such devices, for example, are disclosed in U.S. Pat. Nos. 4,627,429; 4,784,857; 5,662,925; 5,788,983; and 6,113,940, which are all incorporated herein by reference. Characteristically, these devices contain a drug-impermeable backing layer which defines the outer surface of the device and a permeable skin-attaching membrane, such as an adhesive layer, sealed to the barrier layer in such a way as to create a reservoir between them in which the therapeutic agent is placed. In one embodiment of the present invention, policosanol is introduced into the reservoir of a transdermal patch.

4. Kits

A therapeutic composition of the present invention may be packaged in any convenient, appropriate packaging. The present invention therefore further provides kits having one or more containers comprising a therapeutic composition of the present invention and a suitable excipient as described herein and a set of instructions, generally written instructions although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

The invention is further illustrated by the following non-limiting example. All scientific and technical terms have the meanings as understood by one with ordinary skill in the art. The specific examples which follow illustrate the methods in which the compositions of the present invention may be prepared and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLE 1

Tablets comprising a composition of the present invention are prepared as set out in Table III.

TABLE III

| Ingredient | amt/cap | function |
|---|---|---|
| Policosanol | 20 mg | active |
| Calcium phosphate | 261.7 mg | base |
| Cellulose | 49.4 mg | tablet coating agent |
| Stearic acid | 23.8 mg | lubricant |
| Magnesium stearate | 6.8 mg | lubricant |
| Silicon dioxide | 9.4 mg | diluent |

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

The embodiments of the invention in which an exclusive property or privilege is claimed are defined as follows:

1. A method for treating, ameliorating, or inhibiting the development of an inflammatory condition of the gastrointestinal tract, comprising administering to a subject suffering from or at risk of developing said inflammatory condition of the gastrointestinal tract a therapeutically effective amount of a pharmaceutical composition policosanol.

2. The method of claim 1 wherein said policosanol comprises at least one higher primary aliphatic alcohol selected from straight chain primary aliphatic alcohols having 20 to 36 carbon atoms.

3. The composition of claim 2 wherein said policosanol comprises 1-tetracosanol, 1-hexacosanol, 1-octacosanol, 1-triacontanol, 1-dotriacontanol and 1-tetratriacontanol.

4. The composition of claim 3, wherein said policosanol has the following quantitative composition:

| | |
|---|---|
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 5-10% |
| 1-octacosanol (C₂₈) | 10-20% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |

5. The method of claim 1, wherein said inflammatory disorder of the gastrointestinal tract is selected from the group consisting of colorectal cancer, neoplasm of the colon, neoplasm of the rectum, ulcerative colitis, adenomatous polyps, familial polyposis, stomach cancer, and combinations thereof.

6. The method of claim 1, wherein said composition is administered by parenteral, transdermal, intranasal, sublingual, transmucosal, intra-arterial, or intradermal administration.

7. The method of claim 1, wherein said composition is delivered to said subject as a controlled release composition.

8. The method of claim 7, wherein said controlled release composition comprises a flowable thermoplastic polymer composition comprising a biocompatible polymer, a biocompatible solvent, and policosanol, and is delivered to a bodily tissue or fluid in said subject, wherein the amounts of the polymer and the solvent are effective to form a biodegradable polymer matrix containing policosanol in situ when said composition contacts said bodily fluid tissue or fluid.

9. The method of claim 8, wherein said polymer is a poly(alkylene glycol) or a polysaccharide.

10. The method of claim 7, wherein the composition further comprises a controlled release additive.

11. The method of claim 8, wherein said biocompatible polymer is selected from the group consisting of polylactides, polyglycolides, polyanhydrides, polyorthoesters, polycaprolactones, polyamides, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyacrylates, polyalkylene succinates, poly(malic acid), poly(amino acids) and copolymers, terpolymers, cellulose diacetate, ethylene vinyl alcohol, and copolymers and combinations thereof.

12. The method of claim 8, wherein the polymer matrix releases said policosanol by diffusion, erosion, or a combination of diffusion or erosion as the polymer matrix biodegrades in said subject.

13. The method of claim 8, wherein said policosanol is added to said polymer composition prior to administration such that said solid polymer matrix further contains said policosanol.

14. The method of claim 7, wherein said controlled release composition is in film form.

15. The method of claim 14, wherein said film comprises polylactic acid, polyglycolic acid and mixtures and copolymers thereof.

16. The method of claim 7, wherein said controlled release composition is in tablet form.

17. The method of claim 1, further comprising administering at least one chemotherapy drug, wherein said policosanol and said chemotherapy drug are administered as an admixture, separately and simultaneously, or separately in any order.

18. A kit comprising a first container comprising a controlled release formulation of policosanol, said formulation comprising an amount of policosanol effective to treat, ameliorate, or prevent the development of inflammatory disorder of the gastrointestinal tract.

19. The kit of claim 18, further comprising a puncture needle or catheter.
